(19) United States
(12) Patent Application Publication
(10) Pub. No.: US 2006/0111695 A1
(43) Pub. Date: May 25, 2006

(54) Medical device for generating a jet composed of a gas and a liquid

(76) Inventors: Albert Geisser, Luzern (CH); Bruno R. Kezmann, Steinhausen (CH)

Correspondence Address: OSTROLENK FABER GERB & SOFFEN, 1180 AVENUE OF THE AMERICAS, NEW YORK, NY, 100368403, US

(21) Appl. No.: 10/997,347

(22) Filed: Nov. 24, 2004

(51) Int. Cl. A61B 17/00; A61F 5/04

(52) U.S. Cl. 606/1; 606/53

(57) ABSTRACT

A medical cleaning device for cavities of bones is provided in the form of a handheld device and includes a gas reservoir. From this reservoir a first gas stream is taken that is used to propel a cleaning liquid towards an outlet of the device. A second gas stream is taken from the gas of the reservoir and is passed to a mixer wherein the propelled liquid is enriched with gas from the second gas stream before reaching the outlet.

### BACKGROUND OF THE INVENTION

The present invention relates to a medical device or apparatus for generating a jet of liquid or stream of liquid, respectively, which is composed of a liquid and a gas.

A medical device of this kind can be used during surgery to clean the operation field or implants and in particular can be used to clean bone material from bone cavities during prosthesis surgery, for example hip prosthesis surgery.

1. Prior Art

JP-A-2002 059044 shows a multi-purpose portable cartridge type sprayer. This device uses a single gas stream from a gas containing cartridge to feed or propel, respectively, a liquid from a reservoir to an outlet of the device where the liquid forms a spray. Such a device is not well suited for cleaning during surgery since this device provides a spray.

2. Brief Summary of the Invention

Hence, it is a general object of the invention to provide a device which is well suited for cleaning during surgery.

Now, in order to implement this and still further objects of the invention, which will become more readily apparent as the description proceeds, the present invention provides a device which emits a jet of liquid propelled by a first stream of gas and which mixes a second stream of gas to the liquid. It has been found that the jet of gas enriched liquid as provided by the device according to the invention gives a good cleaning action.

In another aspect of the invention, the gas for providing the first and the second gas stream is held under pressure in a common gas reservoir and in particular the gas is provided in a gas cartridge which is removably adapted to the device.

In yet another aspect of the invention, the liquid is provided in a liquid reservoir which is removably adapted to the device.

Preferably the gas cartridge is a CO₂ containing cartridge.

In yet another aspect of the invention, the mixing means is provided with a hollow body being in contact on its outside with the propelled liquid and being provided with a gas inlet into the hollow body for the second stream of gas and with a multitude of openings in the hollow body for feeding gas of the second gas stream into the propelled liquid.

Preferably the hollow body is provided on its outside with elements lowering the speed of flow of the propelled liquid.

In yet another aspect of the invention, a gas stream generating means is provided for generating the gas streams from the reservoir and the gas stream generating means is removable together with the reservoir from the device.

Preferably the gas stream generating means is provided with a safety lock preventing an unwanted activation of the gas and further preferred the gas stream generating means allows removal of the gas cartridge only when the gas pressure therein is lower than a preselected pressure level.

In yet another aspect of the invention, the medical device for generating a jet composed of a liquid and a gas provides a first stream of the gas which propels the liquid from a liquid reservoir towards an outlet of the device and provides a mixing means between the reservoir and the outlet for mixing a second stream of gas with the gas propelled liquid which is then ejected as the jet through the outlet and wherein the mixing means is provided with a hollow body being in contact with its outside with the propelled liquid and being provided with a gas inlet into the hollow body for the second stream of gas and with a multitude of openings in the hollow body for feeding gas of the second gas stream into the propelled liquid.

Preferably the gas is provided in a gas cartridge which is removably adapted to the device and further preferred the liquid is provided in a liquid reservoir which is removably adapted to the device.

Further preferred is that the hollow body is provided on its outside with elements lowering the speed of flow of the propelled liquid.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 is a schematic representation of a device according to the invention and shows as well different gas stream generating means;

FIG. 2 shows the mixing means in greater detail but still in a schematic representation;

FIG. 3 shows another embodiment of the mixing means in a schematic representation;

FIG. 4 shows an embodiment of the gas stream generating means in a schematic representation and in a first state;

FIG. 5 shows the gas stream generating means of FIG. 4 in a second state;

FIG. 6 shows the gas stream generating means of FIGS. 4 and 5 in a third state; and

FIG. 7 shows the gas stream generating means of FIGS. 4, 5 and 6 in a fourth state.

### DETAILED DESCRIPTION OF THE INVENTION

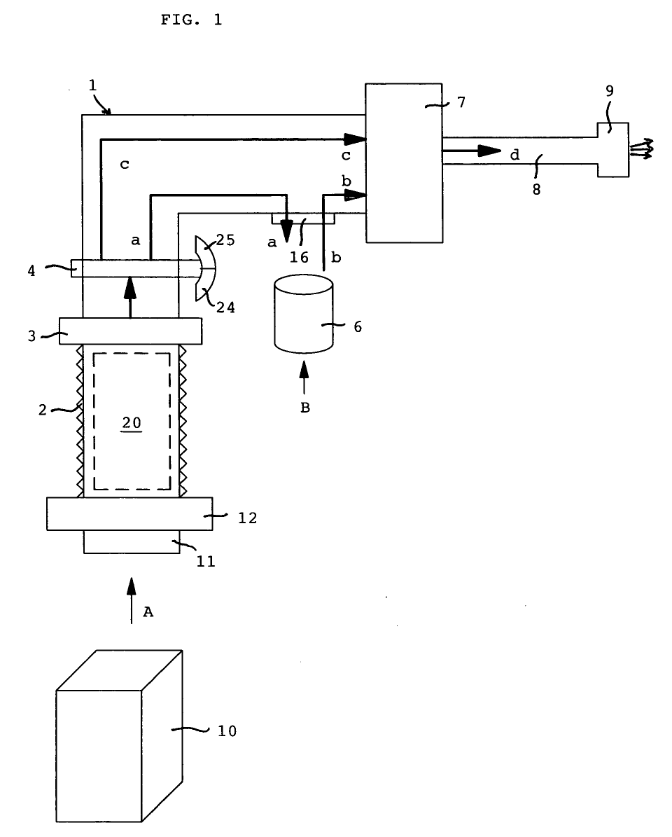

FIG. 1 shows a schematic representation of a device according to the invention. The device is shown as a hand held device with a handle 2 and with a hand operatable valve 4 which activates and deactivates the jet of liquid which is ejected from nozzle 9. The nozzle 9 can be of any known kind and may provide different kind of jets depending on nozzle construction and more or less focussed jets of the liquid as is known for the man skilled in the art. The device 1 can for example be used to clean cavities in bones during surgery and in particular hip prosthesis surgery. This is of course only a preferred use of the device and the cleaning device 1 can be used for other purposes as well where a jet of cleaning liquid is used. The liquid itself can be fed to the device by a line but is preferably contained in a container 6 which can be coupled to the device 1 by coupling 16 on the device and a corresponding coupling means on the container 6. The container may comprise a saline solution 13 and may be a single use sterile packed container which is discarded when the container has been emptied. The device 1 empties the container by a first stream of gas in any known kind by pressing the liquid in container 6 out of the container by the stream of gas which is generated as explained later. It is of course well-known to the man skilled in the art to propel a liquid in this way by a stream of gas as for example shown in the mentioned Japanese document 2002 059044. Any other way of pressing the liquid out of the container by a gas stream or sucking the liquid out of the container by the gas stream can be used as well as is easily apparent to the man skilled in the art. The gas stream which is used to propel the liquid out of the container can be a gas stream coming from a gas cartridge 20 which can be inserted into the handle 2 whereby the cartridge is opened and the pressurized gas contained in the cartridge 20 is fed for example to a pressure reducing valve 3. From this valve 3 the gas is led to a valve 4. This valve 4 can be operated by hand and allows to activate the gas stream towards the container 6 or to deactivate this gas stream in order to activate and deactivate the jet of liquid leaving nozzle 9. The valve 4 can be any kind of valve that allows to block and deblock a gas stream and such valves are well-known to the man skilled in the art. The gas streams are shown in the Figures as thick arrows which represent the gas streams as well as the conduits (pipes, lines, hoses) containing the gas streams in the device itself and which are well known in construction by the man skilled in the art. Accordingly the arrow between the pressure reducing valve 3 and the hand operatable start/stop valve 4 represents a gas stream from cartridge 20 and a conduit connecting valve 3 and valve 4.

According to the invention the liquid that is propelled from container 6 is mixed with a second stream of gas before it leaves the device 1 in form of a liquid jet. To this end a mixing means 7 is provided which is arranged between the container 6 and the outlet or nozzle 9 respectively of the device 1. A second stream of gas is led directly to this mixing means where a mixing of the propelled liquid and the second stream of gas takes place. In FIG. 1 this situation is represented by an arrow c which represents a gas line that leads from valve 4 directly to the mixing means 7 while another gas line a leaves valve 4 and leads to the coupling 16 where it can enter container 6 with the liquid 13 below gas filled space 13′. From this container 6 another line b is shown which leads the liquid propelled by the gas fed by line a into the container to the mixing means 7. From this mixing means the liquid is propelled through conduit 8 to nozzle 9 as shown with arrow d. Both lines a and c can be activated simultaneously by valve 4 so that each time the propelling gas stream is activated and led through line a into the container the second gas stream c is activated as well and led directly to the mixing means 7.

It may as well be that gas stream a for propelling the liquid may be activated without activating the second gas stream c which is represented in FIG. 1 by the divided operating knob 24, 25 of valve 4 by which it shall be represented that the valve may selectively start/stop gas stream a alone or gas streams a and c together. If only the lower part 24 of this knob is pressed then only the propelling gas stream though line a is generated so that a liquid jet through nozzle 9 is generated which has no additional gas added by line c and the mixing means 7 since line c is still blocked by valve 4; a one-way valve in line c (not shown) prevents in this case the filling of line c with liquid. If on the other hand the knob of valve 4 is operated by pressing on part 25 both lines a and c are fed with gas coming out of the pressure reducing valve 3 so that the jet ejected from nozzle 9 consists of liquid that has been propelled by a first gas stream through line a and has been mixed with the second gas stream being fed through line c to the mixing means 7. Such an embodiment allows the user of the device to select whether to start and stop the first gas stream independently of said second gas stream or to start and stop both gas streams together so that he can work with a liquid jet with no additional gas added in the mixing means or with a liquid jet with the gas added, depending on the special cleaning needs that may occur.

Another embodiment is shown in FIG. 1 as well wherein a gas stream generating means 10 can be coupled to the device 1 by coupling 11 as shown with arrow A in FIG. 1. In this way the gas stream generating means replaces the cartridge 20 described beforehand. In another embodiment the gas stream generating means 10 could be inserted into handle 2 instead of being coupled at the end of the handle 2 as shown. A decoupling stop 12 may be provided which prevents a decoupling of gas stream generating means as long as this means generates gas pressure.

The preferred gas used for medical purposes of device 1 is carbon dioxide (CO₂). Depending on the way in which the liquid in container 6 is propelled by the carbon dioxide gas stream a the liquid contains already more or less gas when it is leaving the container 6 and enters the mixing means 7 by line b. However, if the second gas stream is activated additional gas will be added to the propelled liquid in mixing means 7.

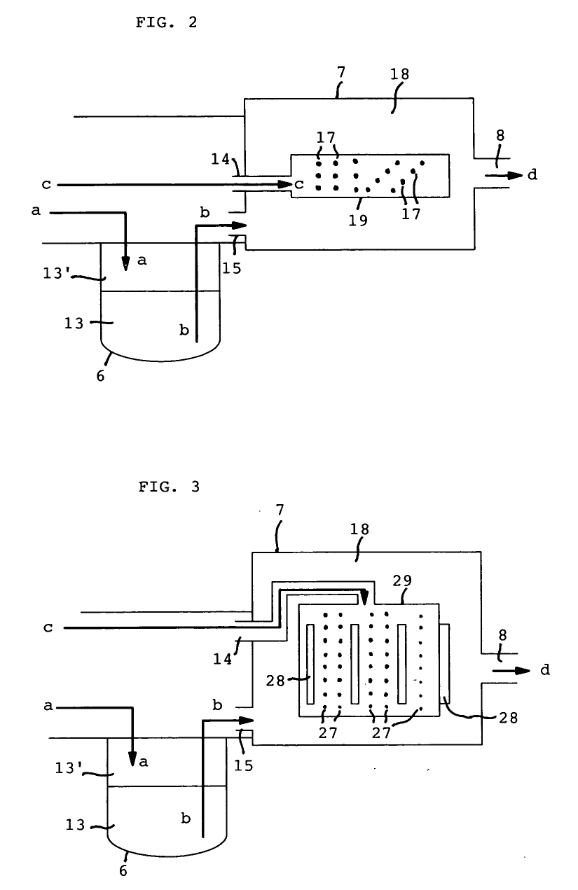

FIG. 2 shows a first embodiment of a mixing means 7. This means 7 has a closed housing except for the inlets 14, 15 and an outlet via conduit 8. A hollow body 19 is held in the housing by fixation means that are not shown. This hollow body 19 is closed at its end facing line 8 and has an inlet 14 where line c for the second gas stream enters the hollow body. The body is closed except for a great number of holes 17 which can be arranged for example circularly or spiraly around the wall of the body and which allow the gas that enters the body by line c to pass out of the body 19 into chamber 18 of the mixing means 7. Into this chamber 18 the liquid propelled by the first gas stream through line a and leaving the container 6 through line b is fed by inlet 15 of the mixing means so that the chamber 18 is filled with the propelled liquid that enters the chamber 18 under pressure through inlet 15 and leaves the chamber 18 through line 8 towards the outlet (nozzle 9) of the device as the liquid jet. Within chamber 18 the propelled liquid is mixed with the gas of the second stream c leaving the body 19 through the small openings 17 therein. Body 19 can be shaped circular in cross section or rectangular or may have any other shape.

FIG. 3 shows another embodiment wherein same parts are shown with same reference numerals as before. In this case a cylindrical hollow body 29 is fixed in chamber 18 of mixing means 7 and the inlet 14 for gas stream c is on the upper side of hollow body 29 which is as well closed except for small holes 27 which allows the gas fed into the body 29 though line c to escape by these holes. Lines a and b are as well provided for propelling the liquid 13 by first gas stream a into chamber 18 where the second gas stream c leaving body 29 through holes 27 is mixed with this propelled liquid and leaves mixing means by line 8 as explained before. In this case as well as in the other embodiment shown before there may be projections 28 within chamber 18 that slow down the liquid propelled by the first gas stream in order to allow a better mixing with the second gas stream. These projections may be present on body 29 itself or may be provided on the inside of the housing of mixing means 7 or may be provided on both.

In the following the special embodiment with the gas generating means 10 is explained but most of the features present in gas generating means could as well be incorporated into handle 2 and be employed together with cartridge 20. As said before the generating means 10 can be coupled to the device 1 by coupling 11 on the device and a corresponding coupling part on the generating means 10.

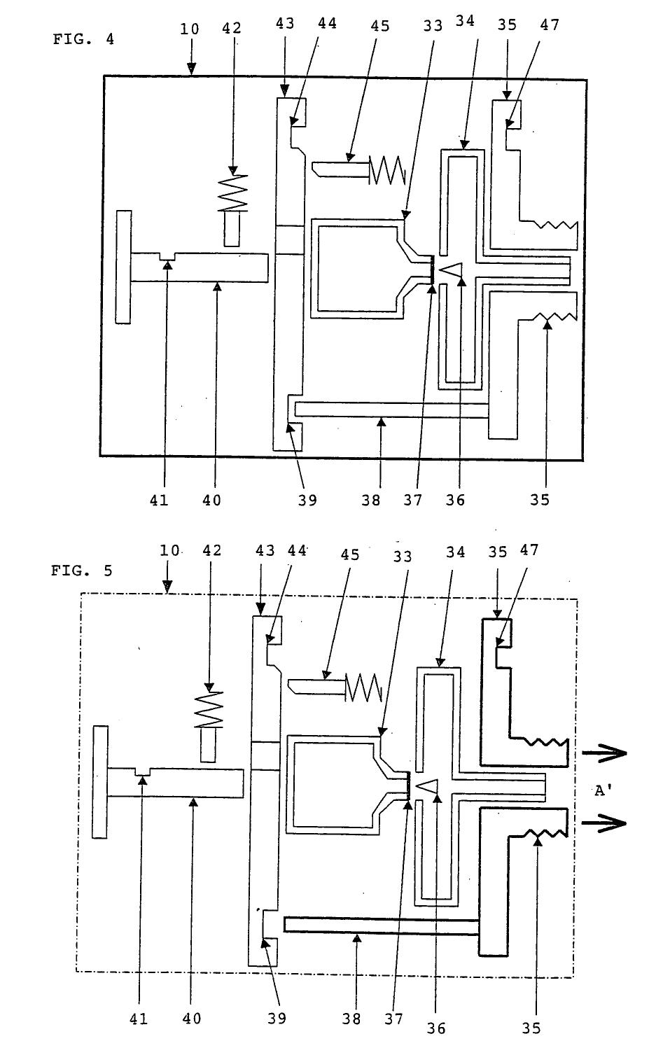

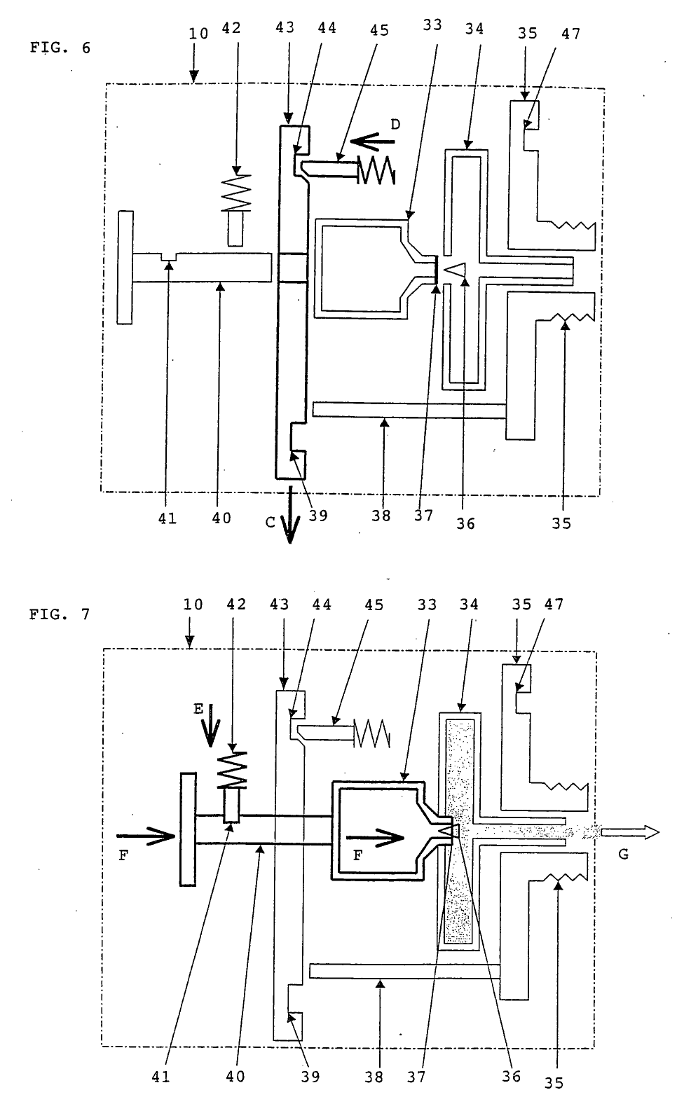

FIG. 4 shows a very schematic representation of gas generating means 10 which contains a gas reservoir 33 which may be a cartridge containing gas, preferably CO₂, either under high pressure or in liquid form. Such gas cartridges are known. Cartridge 33 is closed by a membrane 37 or a valve. Means 10 are provided with a coupling 35 for coupling it to device 1 and the coupling may be of a screw type or another known coupling. The coupling may be connected by a blocking bolt 38 with a securing plate 43 which prevents in the position shown that an operating element 40 can move cartridge 33 in the direction of a pin 36 for opening the cartridge. This safety mechanism will be explained further but of course this is only a schematic representation and shall be understood as only one of several possible embodiments that become readily apparent to the man skilled in the art considering the function of the schematically shown elements. FIG. 5 shows the state of the gas generating means 10 when it has been coupled to device 1. Same reference numerals as used before depict same elements which is as well true for FIGS. 6 and 7. By coupling means 10 to device 1 the coupling 35 has been moved in direction of arrows A′ for example by stopping the housing of device 10 only shown by the dash-dotted line by contacting the device 1 and by thus moving coupling 35 towards device 1 when the generating means 10 is screwed to the device. By this movement the blocking bolt 38 has moved away from hole 39 of safety plate 43. This plate 43 can now be moved downwards in FIG. 5. FIG. 6 now shows how this plate 43 has been moved in direction of arrow C what has been done for example by pressing by hand on a part of plate 43 that projects from the housing of means 10. The securing plate 43 can be blocked in its clearing position as shown in FIG. 6 by a bolt 45 which enters another hole 44 in plate 43 by moving in direction of arrow D when the bolt 45 aligns with hole 44. Bolt 45 can be arranged so that a moving back of securing plate 43 into position of FIG. 5 can be effected if the gas generating means shall not be activated.

But if the gas generating means 10 shall be activated now the actuation element 40 is now pressed by hand in direction of arrow F what is shown in FIG. 7. The element 40 can now pass through a hole in plate 43 and move the cartridge 33 in direction to the piercing element 36 destroying thereby membrane 37 or opening the valve of the cartridge so that the gas can leave the cartridge. Element 40 may be fixed by another bolt 42 entering a hole 41 so that the position of the cartridge 33 can not be changed and the gas will positively leave the cartridge only in the wanted direction.

FIG. 7 shows the opened cartridge 33 in connection with a evaporator wherein the liquid gas of the cartridge evaporates. Preferably the evaporator takes the necessary heat from the environment and is not actively heated. Such evaporators are known and essentially comprise a large area heart exchanger. If the gas is stored in cartridge 33 in gaseous form, the heat exchanger is of course omitted and eventually replaced by a first pressure reducing valve (not shown). In the embodiment of FIG. 7 the gas stream leaves the heat exchanger in direction of arrow G and enters device 1 through coupling 11 where gas streams a and c are formed as explained. For a medical device it is preferred if gas generating means is provided as a sterile packed means which is coupled to device 1 when needed. When the gas filling of means 10 is exhausted, the means is removed from device 1 and replaced by a new means 10 if device 1 is used further. Empty means 10 can then be discarded or reused by replacing cartridge 33. If device is used no longer it can be cleaned and for another use a new means 10 and container 6 can be coupled to the device. The explained safety feature that allows opening of the cartridge only when means 10 has been coupled to device 1 prevents an uncontrolled escape of gas. Coupling 35 can further be adapted to allow a removal of means 10 from device 1 only when the cartridge 33 is empty. To this end a member of the decoupling stop 12 of device 1 projects in a hole 47 of coupling 35 as long as decoupling stop 12 detects a gas pressure.

Thus a medical cleaning device for cavities of bones is provided in the form of a handheld device and includes a gas reservoir. From this reservoir a first gas stream is taken that is used to propel a cleaning liquid towards an outlet of the device. A second gas stream is taken from the gas of the reservoir and is passed to a mixing unit wherein the propelled liquid is enriched with gas from the second gas stream before reaching the outlet.

While there are shown and described presently preferred embodiments of the invention, it is to be distinctly understood that the invention is not limited thereto but may be otherwise variously embodied and practiced within the scope of the following claims.

1. A medical device for generating a jet composed of a liquid and a gas wherein a first stream of said gas propels said liquid from a liquid reservoir towards an outlet of said device and a mixing means is provided between said resevoir and said outlet for mixing a second stream of gas with said gas propelled liquid which is then ejected as said jet through said outlet.

2. The device of claim 1 wherein said gas for providing said first and said second gas stream is held under pressure in a common gas reservoir.

3. The device of claim 2 wherein said gas is provided in a gas cartridge which is removably adapted to said device.

4. The device of claim 1 wherein said liquid is provided in a liquid reservoir which is removably adapted to said device.

5. The device of claim 2 wherein said gas cartridge is a CO₂ cartridge.

6. The device of claim 1 wherein said mixing means is provided with a hollow body being in contact with its outside with said propelled liquid and being provided with a gas inlet into the hollow body for said second stream of gas and with a multitude of openings in said hollow body for feeding gas of said second gas stream into said propelled liquid.

7. The device of claim 6 wherein said hollow body is provided on its outside with elements lowering the speed of flow of said propelled liquid.

8. The device of claim 6 wherein the housing of said mixing means is provided on the inside thereof with elements lowering the speed of flow of said propelled liquid.

9. The device of claim 2 wherein a gas stream generating means is provided for generating the gas streams from said reservoir.

10. The device of claim 9 wherein said gas stream generating means is removable together with said reservoir from said device.

11. The device of claim 9 wherein said gas stream generating means is provided with a safety lock preventing an unwanted activation of said gas.

12. The device of claim 10 comprising a decoupling stop whitch allows removal of said said gas stream generating means coupled to the device only when the gas pressure therein is lower than a preselected pressure level.

13. The device of claim 1 wherein said gas is stored in liquid form.

14. The device of claim 1 wherein said first and second gas streams are started and stopped by a valve and said valve allows the user of the device to select whether to start and stop the first gas stream independently of said second gas stream or to start and stop both gas streams together.

15. A medical device for generating a jet composed of a liquid and a gas wherein a first stream of said gas propels said liquid from a liquid reservoir towards an outlet of said device and a mixing means is provided between said resevoir and said outlet for mixing a second stream of gas with said gas propelled liquid which is then ejected as said jet through said outlet and wherein said mixing means is provided with a hollow body being in contact with its outside with said propelled liquid and being provided with a gas inlet into the hollow body for said second stream of gas and with a multitude of openings in said hollow body for feeding gas of said second gas stream into said propelled liquid.

16. The device of claim 15 wherein said gas for both of said gas streams is provided in a gas cartridge which is removably adapted to said device.

17. The device of claim 15 wherein said liquid is provided in a liquid reservoir which is removably adapted to said device.

18. The device of claim 15 wherein said hollow body is provided on its outside with elements lowering the speed of flow of said propelled liquid.

19. A medical device for generating a jet composed of a liquid and a gas wherein a first stream of said gas propels said liquid from a liquid reservoir towards an outlet of said device and a mixing means is provided between said resevoir and said outlet for mixing a second stream of gas with said gas propelled liquid which is then ejected as said jet through said outlet and wherein said mixing means is provided with a hollow body being in contact with its outside with said propelled liquid and being provided with a gas inlet into the hollow body for said second stream of gas and with a multitude of openings in said hollow body for feeding gas of said second gas stream into said propelled liquid and wherein said hollow body is provided on its outside with elements lowering the speed of flow of said propelled liquid.

20. The device of claim 19 wherein said gas is provided in a gas cartridge which is removably adapted to said device and said liquid is provided in a liquid reservoir which is removably adapted to said device.

21. A medical device for generating a jet composed of a liquid and a gas wherein a first stream of said gas propels said liquid from a liquid reservoir towards an outlet of said device and a mixing means is provided between said resevoir and said outlet for mixing a second stream of gas with said gas propelled liquid which is then ejected as said jet through said outlet and wherein said first and second gas streams are started and stopped by a valve and said valve allows the user of the device to select whether to start and stop the first gas stream independently of said second gas stream or to start and stop both gas streams together.
